(19) United States
(12) Patent Application Publication
(10) Pub. No.: US 2023/0024388 A1
(43) Pub. Date: Jan. 26, 2023

(54) MODULAR MILLING ASSEMBLY FOR ORTHOPAEDIC SURGERY

(71) Applicant: WishBone Medical, Inc., Warsaw, IN (US)

(72) Inventors: Reese Myers, Columbia City, IN (US); Mark Figgie, Riverside, CT (US); David Mayman, New York, NY (US); Ritvik Sarkar, Forest Hills, NY (US); Joseph Lipman, New York, NY (US)

(73) Assignee: Wishbone Medical, Inc., Warsaw, IN (US)

(21) Appl. No.: 17/382,833

(22) Filed: Jul. 22, 2021

(51) Int. Cl. A61B 17/17

(52) U.S. Cl. CPC A61B 17/17; A61B 2017/1602

(57) ABSTRACT

A milling assembly for orthopaedic surgery includes: a milling body including a coupling section with a body coupler, a pilot stem extending from the coupling section, an exterior wall, and a window formed in the exterior wall; and a milling frame that is coupled to the milling body. The milling frame includes a frame coupler that is coupled to the body coupler and a reamer guiding section. The reamer guiding section has a reamer slot that is aligned with the window of the milling body when the frame coupler is coupled to the body coupler.

### BACKGROUND OF THE INVENTION

### 1. Field of the Invention

The present invention relates to milling assemblies, and, more particularly, to milling assemblies for orthopaedic surgery.

### 2. Description of the Related Art

Orthopaedic implants, such as hip implants, are implanted with the assistance of reamers, which are used to form spaces in the bone(s) to accept the implants. Many reamers are used in conjunction with milling assemblies, which may also be referred to as jigs or guides, that help the surgeon form the space in the bone(s) with the proper orientation and placement. In this respect, many different milling assemblies with varying angles that can be used for reamers with varying sizes are known. However, many known milling assemblies have large space requirements and are not well suited for surgery performed on smaller patients, such as children, where there is less space available for the milling assembly.

What is needed in the art is a milling assembly that can address some of the issues with known milling assemblies.

### SUMMARY OF THE INVENTION

The present invention provides a milling assembly including a milling frame that is coupled with a milling body so a reamer slot of the milling frame is aligned with a window of the milling body when a frame coupler of the milling frame is coupled to a body coupler of the milling body.

The invention in one form is directed to a milling assembly for orthopaedic surgery including: a milling body including a coupling section with a body coupler, a pilot stem extending from the coupling section, an exterior wall, and a window formed in the exterior wall; and a milling frame that is coupled to the milling body. The milling frame includes a frame coupler that is coupled to the body coupler and a reamer guiding section. The reamer guiding section has a reamer slot that is aligned with the window of the milling body when the frame coupler is coupled to the body coupler.

The invention in another form is directed to a kit for forming a milling assembly for orthopaedic surgery. The kit includes: a milling body including a coupling section with a body coupler, a pilot stem extending from the coupling section, an exterior wall, and a window formed in the exterior wall; and a milling frame configured to couple to the milling body. The milling frame includes a frame coupler that is configured to couple to the body coupler and a reamer guiding section. The reamer guiding section has a reamer slot that is aligned with the window of the milling body when the frame coupler is coupled to the body coupler.

An advantage of the present invention is the milling assembly has a relatively small space requirement and is thus well-suited for surgery performed on small patients.

Another advantage is the milling body and the milling frame can be adjusted in a wide variety of ways for use with different sized reamers to form spaces with different shapes and volumes.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a partial cut-away view of a femur with an exemplary embodiment of a milling body provided according to the present invention disposed therein;

FIG. 2 is a perspective view of the femur in FIG. 1 with an exemplary embodiment of a milling frame provided according to the present invention being brought toward the milling body;

FIG. 3 is a partial cut-away view of the femur in FIGS. 1-2 when the milling frame of FIG. 2 is coupled with the milling body of FIGS. 1-2;

FIG. 4 is a partial cut-away view of the femur in FIG. 3 with a reamer being brought toward a reamer slot of the milling frame;

FIG. 5A is a partial cut-away view of the femur in FIG. 4 as the reamer of FIG. 4 is rotated while held in the reamer slot;

FIG. 5B is a partial cut-away view of the femur in FIG. 5A after the reamer has been moved within the reamer slot;

FIG. 5C is another partial cut-away view of the femur in FIG. 5B;

FIG. 6A is a partial cut-away view of the femur in FIGS. 1-5C after a space has been formed in the femur;

FIG. 6B is a partial cut-away view of the femur in FIGS. 1-6A after an orthopaedic implant has been implanted therein;

FIG. 7 is a perspective view of another exemplary embodiment of a milling frame provided according to the present invention;

FIG. 8 is a perspective view of the milling frame of FIG. 7 being coupled to an exemplary embodiment of a milling body provided according to the present invention;

FIG. 9A is a partial cut-away view of a femur with the milling body and the milling frame of FIG. 8 disposed therein as a reamer is being inserted into a reamer slot of the milling frame;

FIG. 9B is a partial cut-away view of the femur in FIG. 9A after the reamer has been advanced partway in the reamer slot;

FIG. 9C is a partial cut-away view of the femur in FIGS. 9A-9B after the reamer has been advanced further in the reamer slot;

FIG. 10A is a perspective view of an exemplary embodiment of an inserter that may be coupled to the milling body of FIGS. 8-9C to orient the milling body in the femur;

FIG. 10B is a close-up view of a coupling between the inserter and the milling body of FIG. 10A;

FIG. 11A is a perspective view of another exemplary embodiment of a milling frame with a reamer disposed in a reamer slot of the milling frame for plunge milling, provided in accordance with the present invention;

FIG. 11B is a perspective view of the milling frame and a cut-away view of the reamer of FIG. 11A after the reamer has been moved in the reamer slot;

FIG. 12A is a perspective view of the reamer of FIGS. 11A and 11B being advanced toward the reamer slot of the milling frame;

FIG. 12B is a perspective view of the reamer disposed in the reamer slot of FIGS. 11A-12A;

FIG. 12C is a partial cut-away view of a femur with a milling body disposed therein as the reamer and the milling frame of FIG. 12B is brought toward the milling body;

FIG. 12D is a partial cut-away view of the femur in FIG. 12C when the milling frame is coupled with the milling body;

FIG. 13A is a perspective view of another exemplary embodiment of a milling frame with a reamer slot having a reamer disposed therein;

FIG. 13B is a perspective view of the milling frame of FIG. 13A without the reamer;

FIG. 14A is a perspective view of another exemplary embodiment of a milling frame with a reamer slot having a reamer disposed therein;

FIG. 14B is a perspective view of the milling frame of FIG. 14A without the reamer; and

FIG. 14C is another perspective view of the milling frame of FIGS. 14A and 14B with the reamer of FIG. 14A disposed in the reamer slot.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

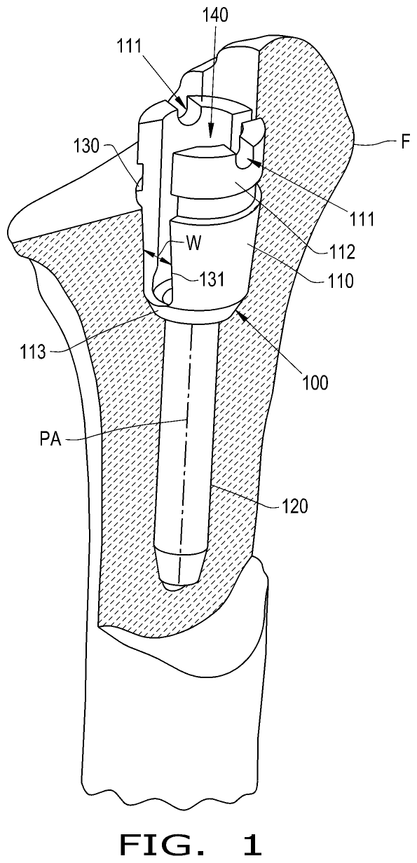

Referring now to the drawings, and more particularly to FIG. 1, a femur F is illustrated that has been partially prepared to accept an exemplary embodiment of a milling body 100 provided according to the present invention. The milling body 100 includes a coupling section 110 with a body coupler 111, which may comprise one or more slots, a pilot stem 120 extending from the coupling section 110, an exterior wall 130, and a window 131 formed in the exterior wall 130. The exterior wall 130 may be part of the coupling section 110 so the window 131 extends into a frame cavity 140 formed in the milling body 100, which will be described further herein. As illustrated, the body coupler 111, if it comprises two slots, may be formed on opposite sides of a top portion 112 of the coupling section 110. The pilot stem 120 may extend from a bottom portion 113 of the coupling section 110, which is opposite the top portion 112. In some embodiments, the pilot stem 120 defines a pilot axis PA and is configured to reside in a pilot opening formed in the femur F. The top portion 112 and the bottom portion 113 of the coupling section 110 may each be generally cylindrically and/or conically shaped about the pilot axis PA. In some embodiments, the window 131 extends from the top portion 112 to the bottom portion 113 so the window 131 extends generally in parallel with the pilot axis PA through almost an entirety of the coupling section 110. A width W of the window 131, which extends in a circumferential direction about the pilot axis PA, may be varied in many different ways to accommodate different widths of reamers. While the window 131 is illustrated as having an open end, in some embodiments the window 131 has no open ends. The milling body 100 may be formed of a variety of suitable materials, including but not limited to metals such as stainless steel, titanium, and/or aluminum, and/or polymers such as polyether ether ketone (PEEK) and/or polyphenylsulfone.

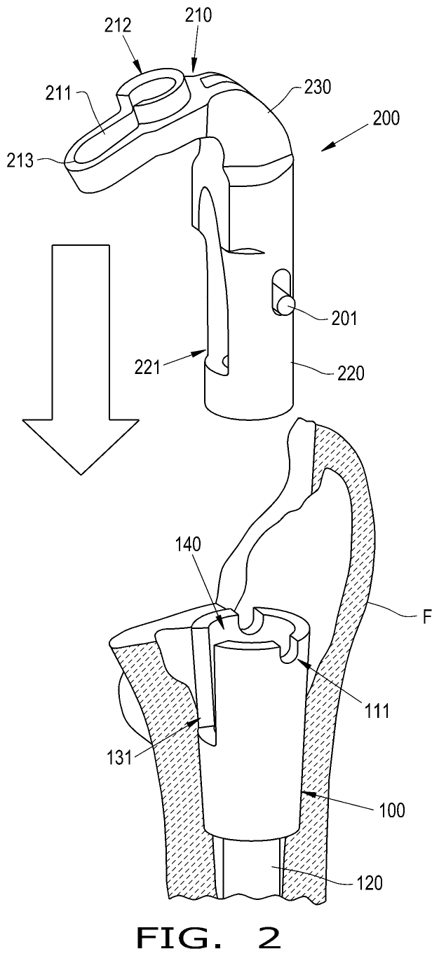

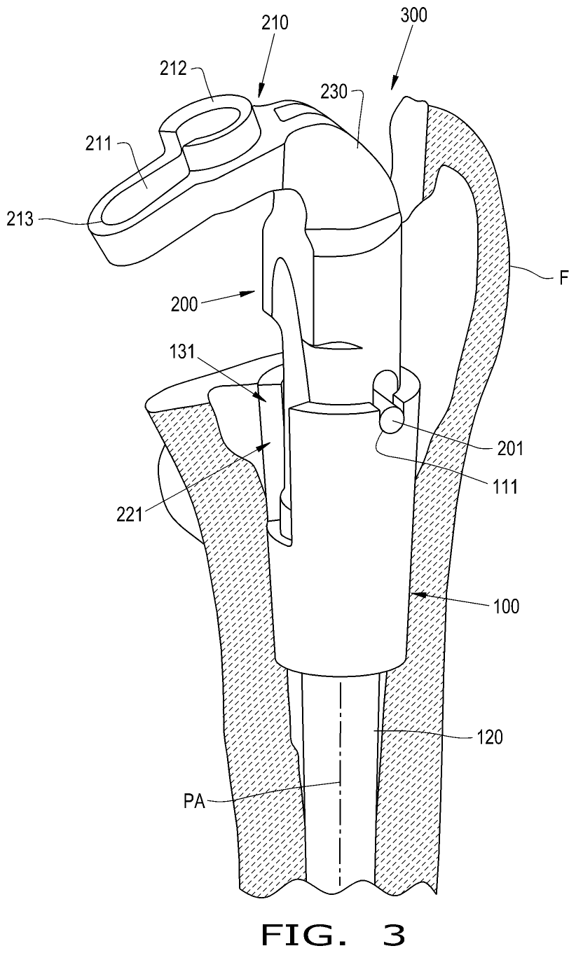

Referring now to FIGS. 2-3, an exemplary embodiment of a milling frame 200 provided according to the present invention is illustrated that is coupled to the milling body 100 to form a milling assembly 300. The milling frame 200 includes a frame coupler 201, which may comprise one or more protrusions, that is configured to couple with the body coupler 111 of the milling body 100 and a reamer guiding section 210. In some embodiments, the frame coupler 201 being coupled to the body coupler 111, as illustrated in FIG. 3, couples the milling frame 200 to the milling body 100 to form the milling assembly 300. While the frame coupler 201 is illustrated as comprising one or more protrusions and the body coupler 111 is illustrated as comprising one or more slots, it should be appreciated that the frame coupler 201 can comprise one or more slots and the body coupler 111 can comprise one or more protrusions. In this respect, the frame coupler 201 and the body coupler 111 can be configured in any suitable way to couple to one another.

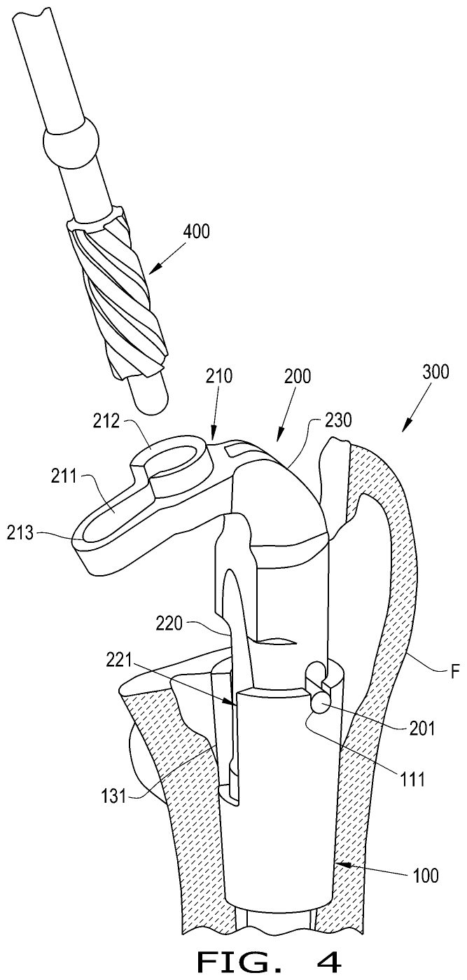

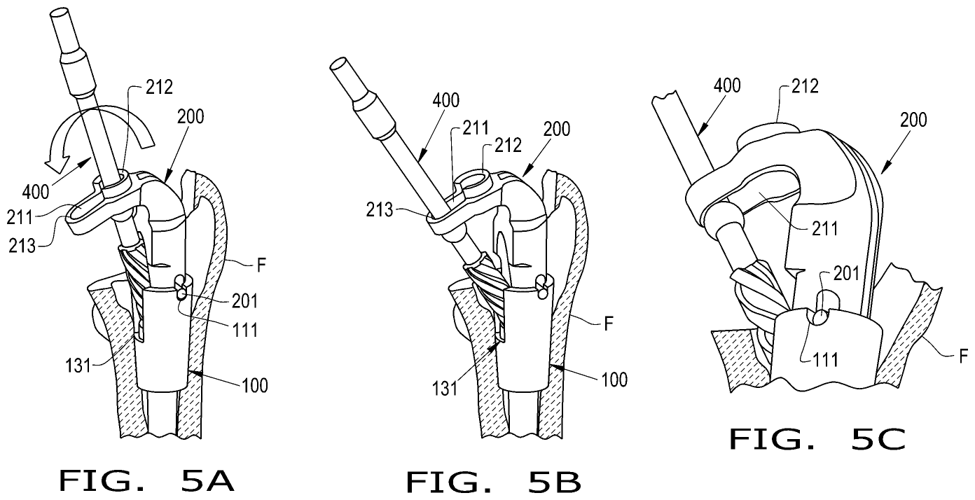

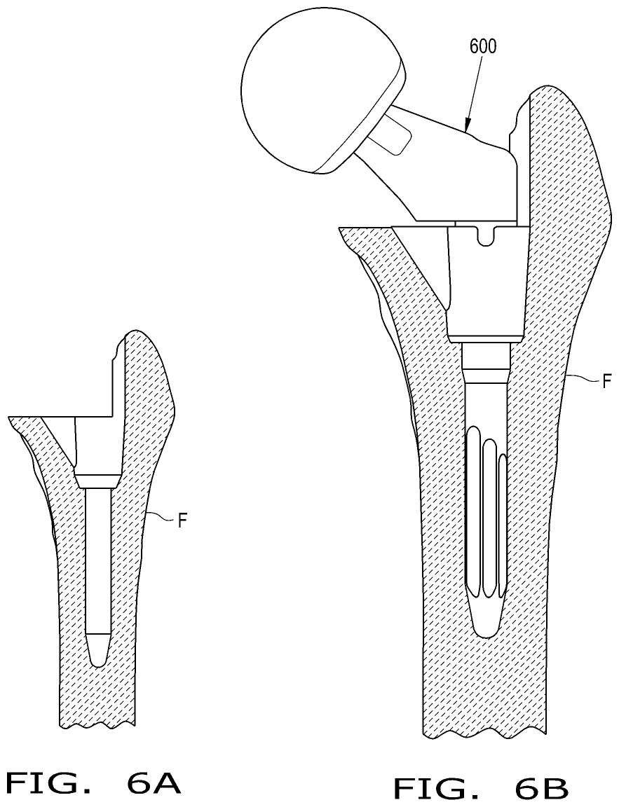

The reamer guiding section 210 has a reamer slot 211 that is aligned with the window 131 of the milling body 100 when the frame coupler 201 is coupled to the body coupler 111. As illustrated in FIGS. 4, 5A, 5B, and 5C, the reamer slot 211 being aligned with the window 131 makes it so a reamer 400 disposed in the reamer slot 211 is directed at the window 131 and can be used to form an angled cut in the femur F, as illustrated in FIG. 6A. In this respect, coupling the frame coupler 201 to the body coupler 111 positions and orients the reamer slot 211 so the reamer 400 rotating within the reamer slot 211 reams tissue at an angle toward the window 131. The window 131 thus signifies to a surgeon where the angled cut in the femur F will be formed when the milling frame 200 couples to the milling body 100 and the reamer 400 is rotated in the reamer slot 211, allowing the surgeon to know precisely where the angled cut in the femur F will be made based on the position of the window 131 in the femur F. The surgeon can thus use the formed milling assembly 300 to form the desired space in the femur F (illustrated in FIG. 6A) to accommodate an orthopaedic implant 600 that has an angled portion, as illustrated in FIG. 6B.

In some embodiments, the milling frame 200 includes a placement section 220 that is configured to be placed in the frame cavity 140 of the milling body 100. The placement section 220 may be similarly shaped and sized to the frame cavity 140 so the placement section 220 has little room to move within the frame cavity 140, reducing the risk of the milling frame 200 being able to move relative to the milling body 100 during use. The placement section 220 may, for example, extend in parallel with the pilot axis PA. The reamer guiding section 210 may be coupled to the placement section 220 and arced with respect to the pilot axis PA, as illustrated. However, it should be appreciated that the reamer guiding section 210 and/or the reamer slot 211 do not need to be arced with respect to the pilot axis PA. The milling frame 200 may include a curved neck 230 where the reamer guiding section 210 meets the placement section 220. In some embodiments, a curvature of the neck 230 controls the arc of the reamer guiding section 210 relative to the pilot axis PA. The placement section 220 may also include a frame window 221 that at least partially overlaps with the window 131 of the milling body 100 when the milling frame 200 is coupled to the milling body 100. Overlapping some or all of the frame window 221 with the window 131 can allow the reamer 400 to be moved into the milling frame 200 and the milling body 100 during reaming, which can reduce the risk of the reamer 400 being forced through either one of the milling body 100 or the milling frame 200 and reaming more tissue than desired.

Referring now to FIGS. 5A, 5B, and 5C, it is illustrated how the reamer 400 may be used to form the angled cut in the femur F. As illustrated, the reamer slot 211 may have an entryway 212 that is raised relative to the rest of the reamer slot 211. The entryway 212 may, for example, be in the shape of an open ring and provide further support for the reamer 400 as the reamer 400 initially cuts into the tissue of the femur F. Upon making the initial cut, the reamer 400 can be advanced in the reamer slot 211 toward a slot end 213 opposite the entryway 212. As the reamer 400 is advanced in the reamer slot 211, the reamer 400 continues to rotate and ream tissue of the femur F.

Once the reamer 400 has been advanced to the slot end 213 while rotating in the reamer slot 211, the angled cut can be complete and the reamer 400 can be removed. The milling assembly 300 can then be removed and the orthopaedic implant 600 implanted into the femur F. In some embodiments, the milling frame 200 is reversibly coupled to the milling body 100 so the milling frame 200 can be uncoupled from the milling body 100 and removed before the milling body 100 is removed from the femur F. The milling frame 200 can be uncoupled from the milling body 100, for example, by uncoupling the frame coupler 201 from the body coupler 111 and removing the placement section 220 from the frame cavity 140. The milling body 100 may then be pulled out of the femur F. Alternatively, the milling frame 200 can be left coupled to the milling body 100 and both the milling frame 200 and the milling body 100 can be removed from the femur F together.

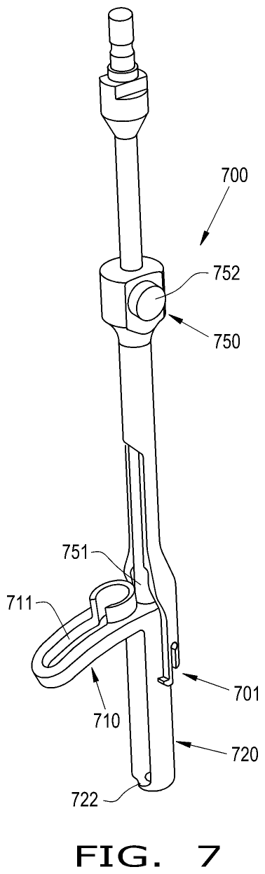

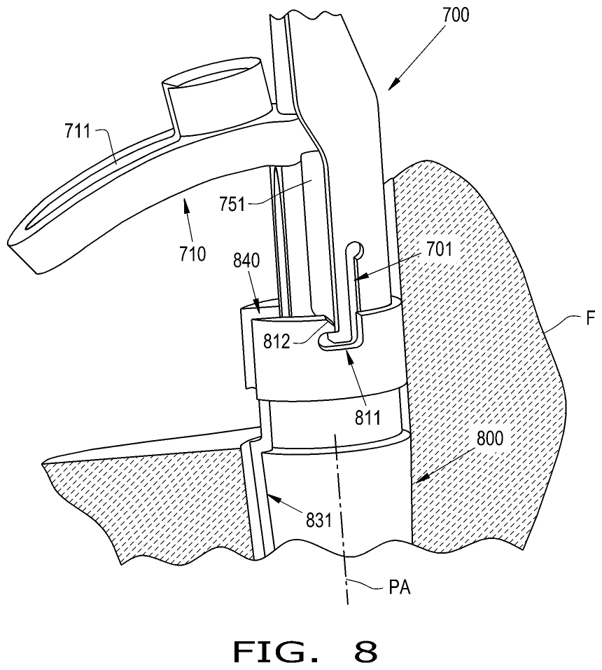

Referring now to FIGS. 7-8, another exemplary embodiment of a milling frame 700 provided according to the present invention is illustrated. Similarly to the previously described milling frame 200, the milling frame 700 is configured to couple to a milling body 800 and includes a frame coupler 701 that is configured to couple to a body coupler 811 of the milling body 800 and a reamer guiding section 710. The reamer guiding section 710 includes a reamer slot 711 that is aligned with a window 831 of the milling body 800 when the frame coupler 701 is coupled to the body coupler 811. The milling body 800 may be similar to previously described milling body 100, with the primary difference being a configuration of the body coupler 811, as will be described further herein.

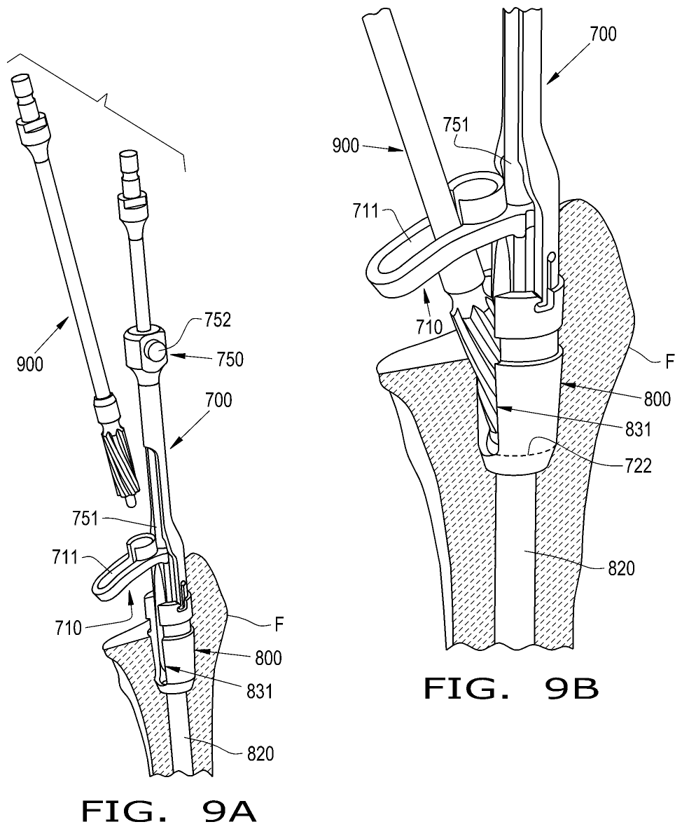

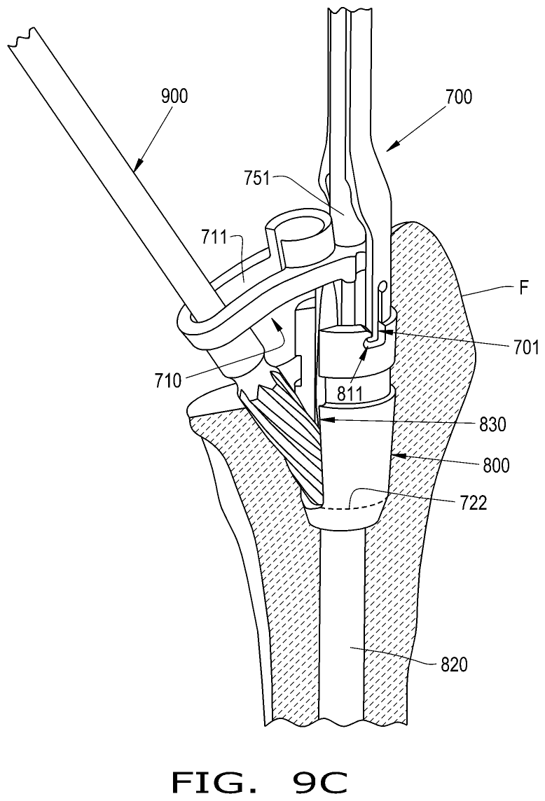

The milling frame 700 includes a placement section 720 that is configured to be disposed in a frame cavity 840 of the milling body 800. The placement section 720 may extend in parallel with a pilot axis PA defined by a pilot stem 820 of the milling body 800 (illustrated in FIGS. 9A and 9B). The reamer guiding section 710 may couple to the placement section 720 so that the reamer guiding section 710 is arced with respect to the pilot axis PA when the frame coupler 701 is coupled to the body coupler 811, but does not meet the placement section 720 at a curved neck like the previously described reamer guiding section 210. Rather, the reamer guiding section 710 may be displaceable with respect to an end 722 of the placement section 720 such that the reamer slot 711 defines an adjustable vertical position relative to the window 831 when the milling frame 700 is coupled to the milling body 800.

To adjust the vertical position of the reamer slot 711, and referring now to FIGS. 9A, 9B, and 9C as well, the reamer guiding section 710 may be coupled to an adjustment assembly 750 that includes a displaceable shaft 751 that is coupled to the reamer guiding section 710 and may be displaced within the milling frame 700. The shaft 751 may be held in position by a button lock 752, which can interlock with the shaft 751 when the button lock 752 is not depressed to prevent the shaft 751 from displacing. As illustrated in FIGS. 9A and 9B, a reamer 900 may be initially placed in the reamer slot 711 and rotated to ream tissue of a femur F, similar to the previously described procedure. After the first pass of reaming is performed, the button lock 752 may be depressed to free the shaft 751 so the shaft 751, and the coupled reamer guiding section 710, can be displaced with respect to the end 722 of the placement section 720, as illustrated in FIG. 9C. After the reamer guiding section 710 has been displaced to the position illustrated in FIG. 9C, the reamer 900 may be rotated in the reamer slot 711 again to cut deeper into the femur F due to the displacement of the shaft 751 and the reamer guiding section 710. Thus, the reamer guiding section 710 of the milling frame 700 can be adjusted using the adjustment 750 to change the final depth of the angled cut that the reamer 900 makes in the femur F using the milling frame 700.

Referring specifically now to FIG. 8, the body coupler 811 of the milling body 800 and the frame coupler 701 of the milling frame 700 are illustrated in greater detail. As illustrated, the body coupler 811 or the frame coupler 701 (the body coupler 811 in the illustrated embodiment) includes a tang slot and the other of the body coupler 811 or the frame coupler 701 (the frame coupler 701 in the illustrated embodiment) includes a deformable tang that is configured to deform as the tang 701 is inserted into the tang slot 811. The tang slot 811 may include a tapered entry surface 812 that the tang 701 rides along as the tang 701 is inserted in the tang slot 811. The tang 701 riding along the tapered entry surface 812 causes the tang 701 to deform until the tang 701 is fully inserted in the tang slot 811, at which point the tang 701 snaps into position to form a snap-fit with the tang slot 811, i.e., where the frame coupler 701 couples to the body coupler 811. The tang 701 is reversibly coupled to the tang slot 811 and may be uncoupled by deforming the tang 701 so the tang 701 can be removed from the tang slot 811 to remove the milling frame 700 from the milling body 800. It should be appreciated that the body coupler 811 can alternatively be formed as a tang and the frame coupler 701 formed as a tang slot. It should be further appreciated that the frame coupler 701 and the body coupler 811 can be configured in a variety of ways to couple to one another.

Referring now to FIGS. 10A and 10B, an exemplary embodiment of an inserter 1000 provided according to the present invention is illustrated for inserting the milling body 800 into a prepared opening formed in the femur F. The milling body 800 may include an inserter slot 860 that is configured to rotatably lock the milling body 800 with the inserter 1000. For example, an inserter pin 1001 of the inserter 1000 may be placed in the inserter slot 860 so the milling body 800 can be rotated by the inserter 1000. By rotating the inserter 1000, a surgeon can also rotate the rotatably locked milling body 800 within the femur F so the window 831 is in position to form the angled cut in the desired orientation. The inserter 1000 thus allows the surgeon to easily re-position the window 831 within the femur F to the desired orientation.

It should be appreciated that while the previously described milling bodies 100, 800 and milling frames 200, 700 are described as being part of a milling assembly for orthopaedic surgery, in some embodiments the milling body 100, 800 and the milling frame 200, 700 are part of a kit for forming a milling assembly for orthopaedic surgery. A kit may include, for example, the previously described milling body 100, 800 and milling frame 200, 700 together in a package, such as a box. The kit may also include a reamer 400, 900 and/or an inserter 1000. The milling body 100, 800 and the milling frame 200, 700 may be packaged together in a sterilized package, such as a sterilized bag, or may be packaged together in separate sterilized containers. In some embodiments, the kit includes a tray that is brought into a surgical environment, such as an operating room, and has the milling body 100, 800 and the milling frame 200, 700 placed therein. In some embodiments, such a kit includes various sizes of the milling body 100, 800 and/or the milling frame 200, 700 so an operating team has a variety of options to use during a surgical procedure. The milling body 100, 800 and the milling frame 200, 700, and other optional components, of such a kit may be coupled together, as previously described, to form the previously described milling assemblies. It should thus be appreciated that, in some embodiments, a kit is provided that includes the milling body 100, 800 and the milling frame 200, 700 as separated parts that are configured to be coupled together in order to form a milling assembly for orthopaedic surgery.

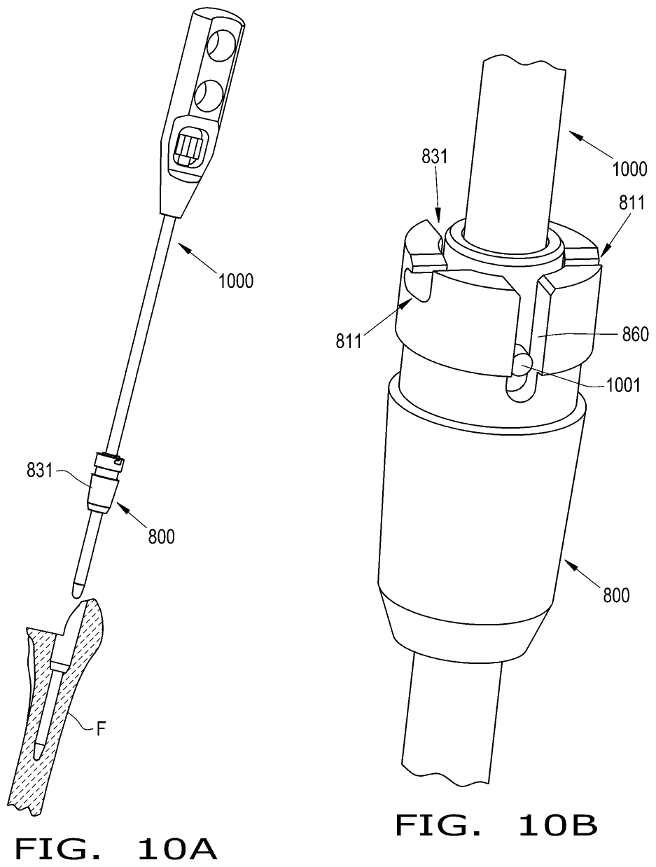

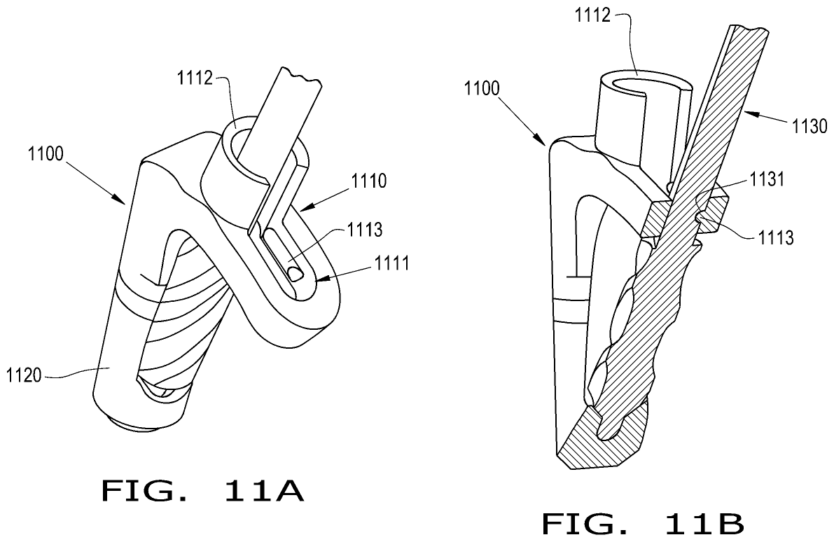

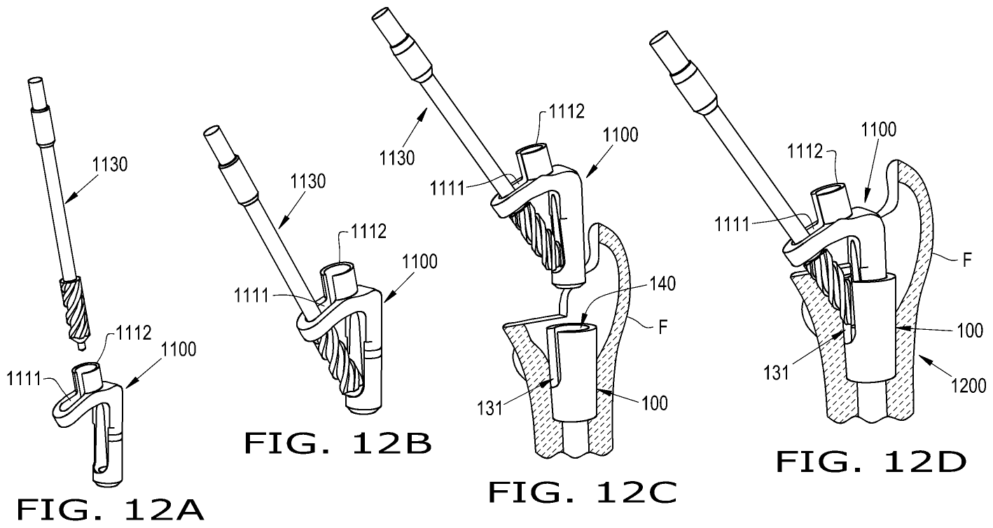

Referring now to FIGS. 11A-12D, another exemplary embodiment of a milling frame 1100 provided according to the present invention is illustrated that can be used to form a milling assembly 1200 with a milling body, such as the previously described milling body 100. As illustrated, the milling frame 1100 includes a reamer guiding section 1110 that is coupled to a placement section 1120 configured to be placed in the frame cavity 140 of the milling body 100. The reamer guiding section 1110 has a reamer slot 1111 with an elevated entryway 1112 that may be in the shape of a partially open cylinder. The reamer slot 1111 may also include one or more protrusions 1113 therein that are configured to fit within a corresponding slot 1131 of a reamer 1130. The protrusions 1113 fitting within the slot 1131 of the reamer 1130 can help hold the reamer 1130 in position as the milling frame 1100 is coupled to the milling body 100, as illustrated in FIGS. 12C and 12D. The milling frame 1100 may be well suited for plunge milling by virtue of the protrusion 1113 holding the reamer 1130 in position as the milling frame 1100 and rotating reamer 1130 are advanced. As the placement section 1120 is advanced in the frame cavity 140, the rotating reamer 1130 reams tissue. It should be appreciated that, in other respects, the milling frame 1100 can be similar to previously described milling frames.

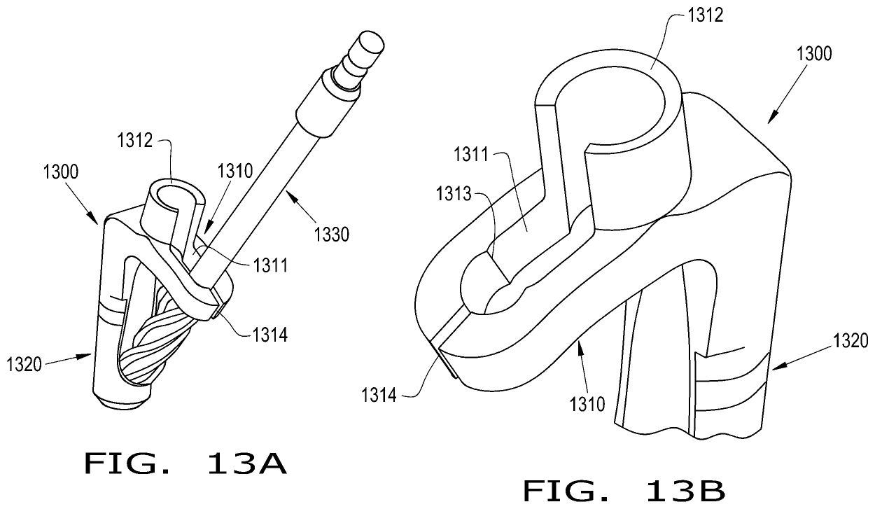

Referring now to FIGS. 13A and 13B, another exemplary embodiment of a milling frame 1300 provided according to the present invention is illustrated. The milling frame 1300 includes a reamer guiding section 1310 that is coupled to a placement section 1320 configured to be placed in a frame cavity 140 of a milling body 100. The reamer guiding section 1310 includes a reamer slot 1311 with an elevated entryway 1312. Unlike the previously described reamer slot 1211, the reamer slot 1311 includes a restriction 1313 that defines an opening smaller than a diameter of a reamer 1330 placed in the reamer slot 1311. The reamer guiding section 1310 also has a split end 1314 opposite the entryway 1312 that allows a partial opening of the reamer slot 1311 when the reamer 1330 is advanced in the reamer slot 1311 from the entryway 1312 toward the split end 1314. When the reamer 1330 abuts the restriction 1313, the reamer 1330 causes opening of the reamer slot 1311 and further advancement of the reamer 1330 to a portion of the reamer slot 1311 between the restriction 1313 and the split end 1314 causes the reamer guiding section 1310 to clamp on the reamer 1330, holding the reamer 1330 in place. In this respect, the milling frame 1300 illustrated in FIGS. 13A and 13B is also well suited for plunge milling due to the holding force exerted on the reamer 1330.

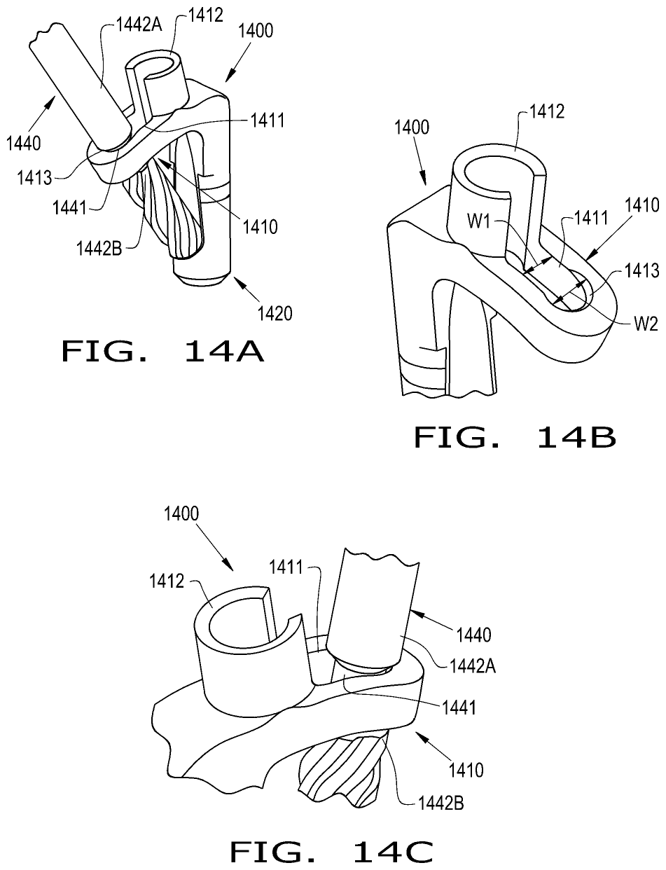

Referring now to FIGS. 14A, 14B, and 14C, another exemplary embodiment of a milling frame 1400 provided according to the present invention is illustrated. The milling frame 1400 includes a reamer guiding section 1410 that is coupled to a placement section 1420 configured to be placed in a frame cavity 140 of a milling body 100. The reamer guiding section 1410 includes a reamer slot 1411 with an elevated entryway 1412. The reamer slot 1411 may define a first slot width W1 adjacent to the entryway 1412 and a second slot width W2 that is adjacent to a slot end 1413 opposite the entryway 1412. The second slot width W2 may be smaller than the first slot width W1 to hold a reamer 1440 inserted in the reamer slot 1411. Particularly, the reamer 1440 may include a narrowed reamer portion 1441 that is sandwiched between two wider portions 1442A, 1442B. The reamer portion 1441 may have a diameter that is approximately the same, if not slightly smaller, than the second slot width W2 while the wider portions 1442A, 1442B have diameters that are both larger than the second slot width W2 but smaller than the first slot width W1. In this respect, the reamer 1440 can be advanced in the reamer slot 1411 so the reamer portion 1441 resides in the part of the reamer slot 1411 with the second slot width W2, with the wider portions 1442A, 1442B preventing the reamer 1440 from being pulled out of the reamer slot 1411 vertically. The wider portion 1442A, for example, can be used to push the milling frame 1400 downwardly for plunge milling. Once the plunge milling is performed, the reamer portion 1441 can be advanced into the portion of the reamer slot 1411 with the first slot width W1 so the reamer 1440 can be removed.

From the foregoing, it should be appreciated that the milling assemblies provided according to the present invention, and the kits for forming such milling assemblies, can be used to predictably form angled cuts in bones with a lower space requirement than known assemblies. By incorporating a window in the milling body, which can correspond to where the angled cut will be formed, the reamer slot of the milling frame being aligned with the window allows the surgeon to know where the angled cut will be when the milling body is positioned in a bone. The reamer slot being arced relative to the pilot axis allows reaming in an arc, as opposed to going down the pilot axis, which can reduce the space requirements of the milling assemblies. Further, the depth of the angled cut can be readily adjusted using differently sized milling frames and/or by using a milling frame with an adjustable reamer section guide. The milling assemblies and kits for forming such milling assemblies provided according to the present invention thus provide a surgeon with precise placement and control for forming an angled cut in a bone using a compact configuration.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A milling assembly for orthopaedic surgery, comprising:
a milling body comprising a coupling section comprising a body coupler, a pilot stem extending from the coupling section, an exterior wall, and a window formed in the exterior wall; and
a milling frame that is coupled to the milling body, the milling frame comprising a frame coupler that is coupled to the body coupler and a reamer guiding section, the reamer guiding section comprising a reamer slot that is aligned with the window of the milling body when the frame coupler is coupled to the body coupler.

2. The milling assembly of claim 1, wherein the frame coupler being coupled to the body coupler couples the milling frame to the milling body.

3. The milling assembly of claim 1, wherein the coupling section comprises the exterior wall.

4. The milling assembly of claim 1, wherein the milling body comprises a frame cavity and the milling frame comprises a placement section disposed in the frame cavity.

5. The milling assembly of claim 4, wherein the placement section comprises an end and the reamer guiding section is displaceable with respect to the end such that the reamer slot defines an adjustable vertical position relative to the window when the milling frame is coupled to the milling body.

6. The milling assembly of claim 4, wherein the placement section extends in parallel with a pilot axis defined by the pilot stem, the reamer guiding section coupling to the placement section and being arced with respect to the pilot axis.

7. The milling assembly of claim 6, wherein the milling frame comprises a curved neck where the reamer guiding section meets the placement section.

8. The milling assembly of claim 4, wherein the placement section comprises a frame window that at least partially overlaps with the window of the milling body when the milling frame is coupled to the milling body.

9. The milling assembly of claim 1, wherein the milling body comprises an inserter slot configured to rotatably lock the milling body with an inserter.

10. The milling assembly of claim 1, wherein the milling frame is reversibly coupled to the milling body.

11. The milling assembly of claim 1, wherein the body coupler or the frame coupler comprises a tang slot and the other of the body coupler or the frame coupler comprises a deformable tang that resides in the tang slot and is configured to deform as the tang is inserted into the tang slot.

12. The milling assembly of claim 1, further comprising a reamer held in the reamer slot and configured to ream biological tissue as the reamer rotates.

13. A kit for forming a milling assembly for orthopaedic surgery, the kit comprising:
a milling body comprising a coupling section comprising a body coupler, a pilot stem extending from the coupling section, an exterior wall, and a window formed in the exterior wall; and
a milling frame configured to couple to the milling body, the milling frame comprising a frame coupler that is configured to couple to the body coupler and a reamer guiding section, the reamer guiding section comprising a reamer slot that is aligned with the window of the milling body when the frame coupler is coupled to the body coupler.

14. The kit of claim 13, wherein the coupling section comprises the exterior wall.

15. The kit of claim 13, wherein the milling body comprises a frame cavity and the milling frame comprises a placement section configured to be disposed in the frame cavity when the frame coupler is coupled to the body coupler.

16. The kit of claim 15, wherein the placement section comprises an end and the reamer guiding section is displaceable with respect to the end such that the reamer slot defines an adjustable vertical position relative to the window when the milling frame is coupled to the milling body.

17. The kit of claim 15, wherein the placement section is configured to extend in parallel with a pilot axis defined by the pilot stem when the milling frame is coupled to the milling body, the reamer guiding section coupling to the placement section and configured to be arced with respect to the pilot axis when the milling frame is coupled to the milling body.

18. The kit of claim 17, wherein the milling frame comprises a curved neck where the reamer guiding section meets the placement section.

19. The kit of claim 15, wherein the placement section comprises a frame window that at least partially overlaps with the window of the milling body when the milling frame is coupled to the milling body.

20. The kit of claim 13, wherein the body coupler or the frame coupler comprises a tang slot and the other of the body coupler or the frame coupler comprises a deformable tang that is configured to deform as the tang is inserted into the tang slot.
